(19) United States
(12) Patent Application Publication
(10) Pub. No.: US 2016/0136170 A1
(43) Pub. Date: May 19, 2016

(54) USE OF A TETRASUBSTITUTED PYRAZOLO[4, 3-D]PYRIMIDINE COMPOUND FOR TREATING DIABETIC NEPHROPATHY

(71) Applicant: Pfizer Inc., New York, NY (US)

(72) Inventors: Valerie Clerin, Watertown, MA (US); Jeremy Gale, Somerville, MA (US); Nihad Tamimi, Canterbury (GB)

(73) Assignee: Pfizer Inc., New York, NY (US)

(21) Appl. No.: 15/002,530

(22) Filed: Jan. 21, 2016

Related U.S. Application Data
(63) Continuation of application No. 14/434,236, filed on Apr. 8, 2015, now abandoned.
(60) Provisional application No. 61/717,429, filed on Oct. 23, 2012.

(51) Int. Cl. A61K 31/519; A61K 45/06

(52) U.S. Cl. CPC A61K 31/519; A61K 45/06

(57) ABSTRACT

The present invention relates to methods of delaying progression to end stage renal disease (ESRD) in patients comprising administration of 1-(2-ethoxyethyl)-5-(ethyl(methyl)amino)-7-((4-methylpyridin-2-yl)amino)-N-(methylsulfonyl)-1H-pyrazolo[4,3-d]pyrimidine-3-carboxamide. The present invention also includes administration of pharmaceutical compositions for delaying progression to ESRD.

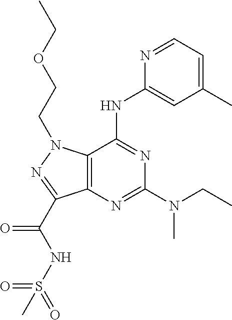 [SMILES: CCOCCn1nc(C(=O)NS(C)(=O)=O)c2nc(N(C)CC)nc(Nc3cc(C)ccn3)c21]

1-(2-ethoxyethyl)-5-(ethyl(methyl)amino)-7-((4-methylpyridin-2-yl)amino)-N-(methylsulfonyl)-1H-pyrazolo[4,3-d]pyrimidine-3-carboxamide

### FIELD OF THE INVENTION

The present invention relates to methods of treating and/or preventing progression of diabetic nephropathy and/or chronic kidney disease using a tetrasubstituted pyrazolo[4,3-d]pyrimidine compound or pharmaceutical compositions containing the tetrasubstituted pyrazolo[4,3-d]pyrimidine compound.

### BACKGROUND OF THE INVENTION

Diabetic nephropathy (DN) is a progressive kidney disease caused by diabetes that affects up to 40% of Type I or Type II diabetic patients. DN is characterized by albuminuria (protein in the urine), progressive decline of renal function leading to end stage renal disease (ESRD), hypertension, and increased cardiovascular morbidity and mortality. ESRD is a life-threatening condition of complete or almost complete loss of kidney function requiring dialysis or kidney transplantation.

The current standard-of-care therapy for patients with diabetic nephropathy targets the Renin Angiotensin System (RAS) using angiotensin converting enzyme (ACE) inhibitors and/or angiotensin II receptor blockers (ARBs) but the use of optimal doses of these drugs is limited by the risk for hyperkalaemia (high serum potassium level), which is a serious side-effect. Hyperkalamia can lead to abnormal heart rhythms and, in extreme cases, death. In view of the modest efficacy of currently available treatments to delay the progression of diabetic nephropathy there is an unmet medical need for compounds, alone or in combination with the current standard of care therapies, that delay patients progressing to end-stage renal disease.

The present invention relates to a new therapeutic approach for treating DN and CKD that targets the NO signalling pathway, distinct from the RAS approach, using the phosphodiesterase type 5 (PDE5) inhibitor, 1-(2-ethoxyethyl)-5-(ethyl(methyl)amino)-7-((4-methylpyridin-2-yl)amino)-N-(methylsulfonyl)-1H-pyrazolo[4,3-d]pyrimidine-3-carboxamide.

### SUMMARY OF THE INVENTION

The present invention relates to methods of delaying progression of CKD, specifically diabetic nephropathy, and/or preventing ESRD in patients comprising the step of administering to the patient, in need of such treatment, a therapeutically effective amount of 1-(2-ethoxyethyl)-5-(ethyl(methyl)amino)-7-((4-methylpyridin-2-yl)amino)-N-(methylsulfonyl)-1H-pyrazolo[4,3-d]pyrimidine-3-carboxamide (Example 1) or a pharmaceutically acceptable salt thereof.

In another embodiment, the present invention relates to methods of delaying progression of CKD, specifically diabetic nephropathy, and/or preventing ESRD in patients comprising the step of administering to the patient, in need of such treatment, a pharmaceutical composition comprising a therapeutically effective amount of 1-(2-ethoxyethyl)-5-(ethyl(methyl)amino)-7-((4-methylpyridin-2-yl)amino)-N-(methylsulfonyl)-1H-pyrazolo[4,3-d]pyrimidine-3-carboxamide, or a pharmaceutically acceptable salt thereof, and at least one pharmaceutically acceptable carrier, diluent, or excipient.

### BRIEF DESCRIPTION OF THE DRAWINGS

FIG. 1 shows that microalbuminuria is significantly decreased in Type II diabetic male patients after treatment with sildenafil citrate administered at 50 mg daily for 30 days (n=20) relative to baseline or Placebo (n=20)).

### DETAILED DESCRIPTION OF THE INVENTION

Nitric oxide (NO) contributes to the maintenance of normal kidney function. NO production and/or availability are decreased in patients with advanced DN. Reduced NO signaling contributes to the development of albuminuria and progression of DN in humans. The more albumin in urine, the faster the progression to ESRD.

Microalbuminuria is defined as a urinary albumin to creatinine ratio (UACR) in men between 30 mg/g and 300 mg/g and macroalbuminuria is defined as a UACR of >300 mg/g. The presence of macroalbuminuria in diabetic nephroapthy best correlates with progression of renal disease.

Chronic kidney disease, also known as chronic renal disease, is a progressive loss of renal function over a period of months or years with a declining glomerular filtration rate (GFR) representing reduced renal function and progression of CKD. The GFR in milliters per minute (mL/min) defines the five stages of CKD: ≧90 mL/min is stage 1; 60-89 mL/min is stage 2; 30-59 mL/min is stage 3; 15-29 mL/min is stage 4; and <15 mL/min is stage 5. Impaired NO signalling has been associated with CKD. This is caused by a combination of reduced NO production, depletion/inactivation of NO by reactive oxygen species and by a dysfunction of soluble guanylate cyclase (sGC). The associated NO deficiency and dysfunctional sGC promotes hypertension and accelerates progression of renal disease.

Inhibiting PDE5 may restore the integrity of the NO signalling pathway, resulting in a number of beneficial effects, including lowering albuminuria and decreasing blood pressure. NO is released from nerve endings and vascular endothelial cells in areas of the cardiovascular system, by the action of shear-stress and local vasoactive agents, such as bradykinin. NO causes smooth muscle relaxation through activation of guanylate cyclase and consequent increase in cyclic guanosine 5′ monophosphate (cGMP). PDE5 specifically degrades cGMP, and an inhibitor of human PDE5 may therefore cause an increase in the levels of cGMP. Elevated cGMP reduces levels of intracellular calcium causing relaxation of smooth muscle cells and ultimately reductions in arterial pressure, vascular resistance, and increased blood flow.

1-(2-ethoxyethyl)-5-(ethyl(methyl)amino)-7-((4-methylpyridin-2-yl)amino)-N-(methylsulfonyl)-1 H-pyrazolo[4,3-d]pyrimidine-3-carboxamide (Example 1) is a selective and competitive inhibitor (IC₅₀=0.71 nM) of human PDE5. Example 1 shows comparable enzyme inhbitory potency against rat PDE5 (IC₅₀=0.93 nM) and functionally potentiates the vasorelaxant action of NO in isolated aorta studies in rats (EC₅₀=3.1 nM). Example 1 exhibits low clearance in dogs and rats, leading to long half-life values, and oral bioavailability is high in both dogs and rats. Example 1 shows no significant inhibition of the major human cytochrome P450 enzymes and is therefore unlikely to significantly alter the metabolism of coadministered drugs that are substrated for these enzymes.

In another embodiment, the present invention relates to methods of treating or preventing progression of diabetic nephropathy in a patient comprising the step of administering to the patient, in need of such treatment, a therapeutically effective amount of 1-(2-ethoxyethyl)-5-(ethyl(methyl)amino)-7-((4-methylpyridin-2-yl)amino)-N-(methylsulfonyl)-1H-pyrazolo[4,3-d]pyrimidine-3-carboxamide or a pharmaceutically acceptable salt thereof.

In another embodiment, the present invention relates to methods of treating or preventing progression of diabetic nephropathy in a patient comprising the step of administering to the patient, in need of such treatment, a pharmaceutical composition comprising a therapeutically effective amount of 1-(2-ethoxyethyl)-5-(ethyl(methyl)amino)-7-((4-methylpyridin-2-yl)amino)-N-(methylsulfonyl)-1H-pyrazolo[4,3-d]pyrimidine-3-carboxamide, or a pharmaceutically acceptable salt thereof, and at least one carrier, diluent or excipient.

In another embodiment, the present invention relates to methods of treating or preventing progression of chronic kidney disease in a patient comprising the step of administering to the patient, in need of such treatment, a therapeutically effective amount of 1-(2-ethoxyethyl)-5-(ethyl(methyl)amino)-7-((4-methylpyridin-2-yl)amino)-N-(methylsulfonyl)-1H-pyrazolo[4,3-d]pyrimidine-3-carboxamide or a pharmaceutically acceptable salt thereof. In particular, the methods of the present invention may be used to treat or prevent CKD stage 3 or 4.

In another embodiment, the present invention relates to methods of treating or preventing progression of chronic kidney disease in a patient comprising the step of administering to the patient, in need of such treatment, a pharmaceutical composition comprising a therapeutically effective amount of 1-(2-ethoxyethyl)-5-(ethyl(methyl)amino)-7-((4-methylpyridin-2-yl)amino)-N-(methylsulfonyl)-1H-pyrazolo[4,3-d]pyrimidine-3-carboxamide, or a pharmaceutically acceptable salt thereof, and at least one carrier, diluent or excipient. In particular, the methods of the present invention may be used to treat or prevent CKD stage 3 or 4.

In another embodiment, the present invention relates to methods of reducing albumin in urine in a patient comprising the step of administering to the patient, in need of such treatment, a therapeutically effective amount of 1-(2-ethoxyethyl)-5-(ethyl(methyl)amino)-7-((4-methylpyridin-2-yl)amino)-N-(methylsulfonyl)-1H-pyrazolo[4,3-d]pyrimidine-3-carboxamide or a pharmaceutically acceptable salt thereof.

In another embodiment, the present invention relates to methods of reducing albumin in urine in a patient comprising the step of administering to the patient, in need of such treatment, a pharmaceutical composition comprising a therapeutically effective amount of 1-(2-ethoxyethyl)-5-(ethyl(methyl)amino)-7-((4-methylpyridin-2-yl)amino)-N-(methylsulfonyl)-1H-pyrazolo[4,3-d]pyrimidine-3-carboxamide, or a pharmaceutically acceptable salt thereof, and at least one carrier, diluent or excipient.

In another embodiment, the present invention relates to methods of treating or preventing macroalbuminuria in a patient comprising the step of administering to the patient, in need of such treatment, a therapeutically effective amount of 1-(2-ethoxyethyl)-5-(ethyl(methyl)amino)-7-((4-methylpyridin-2-yl)amino)-N-(methylsulfonyl)-1H-pyrazolo[4,3-d]pyrimidine-3-carboxamide or a pharmaceutically acceptable salt thereof.

In another embodiment, the present invention relates to methods of treating or preventing macroalbuminuria in a patient comprising the step of administering to the patient, in need of such treatment, a pharmaceutical composition comprising a therapeutically effective amount of 1-(2-ethoxyethyl)-5-(ethyl(methyl)amino)-7-((4-methylpyridin-2-yl)amino)-N-(methylsulfonyl)-1H-pyrazolo[4,3-d]pyrimidine-3-carboxamide, or a pharmaceutically acceptable salt thereof, and at least one carrier, diluent or excipient.

In another embodiment, 1-(2-ethoxyethyl)-5-(ethyl(methyl)amino)-7-((4-methylpyridin-2-yl)amino)-N-(methylsulfonyl)-1H-pyrazolo[4,3-d]pyrimidine-3-carboxamide, or a pharmaceutically acceptable salt thereof, may be employed in combination with another phosphodiesterase type 5 (PDE5) inhibitor including, but not limited to, avanafil, lodenafil, mirodenafil, sildenafil, tadalafil, vardenafil, and udenafil. The combination may be administered separately or within the same pharmaceutical composition.

In another embodiment, 1-(2-ethoxyethyl)-5-(ethyl(methyl)amino)-7-((4-methylpyridin-2-yl)amino)-N-(methylsulfonyl)-1H-pyrazolo[4,3-d]pyrimidine-3-carboxamide, or a pharmaceutically acceptable salt thereof, may be employed in combination with an angiotensin-converting-enzyme (ACE) inhibitor including, but not limited to, captopril, enalapril, lisinopril, perindopril, and ramipril. The combination may be administered separately or within the same pharmaceutical composition.

In another embodiment, 1-(2-ethoxyethyl)-5-(ethyl(methyl)amino)-7-((4-methylpyridin-2-yl)amino)-N-(methylsulfonyl)-1H-pyrazolo[4,3-d]pyrimidine-3-carboxamide, or a pharmaceutically acceptable salt thereof, may be employed in combination with an angiotensin II receptor blocker (ARB) including, but not limited to, losartan, candesartan, valsartan, irbesartan, telmisartan, eprosartan, olmesartan, and azilsartan. The combination may be administered separately or within the same pharmaceutical composition.

In another embodiment, 1-(2-ethoxyethyl)-5-(ethyl(methyl)amino)-7-((4-methylpyridin-2-yl)amino)-N-(methylsulfonyl)-1H-pyrazolo[4,3-d]pyrimidine-3-carboxamide, or a pharmaceutically acceptable salt thereof, may be employed in combination with both an angiotensin-converting-enzyme (ACE) inhibitor and an angiotensin II receptor blocker (ARB). The ACE inhibitor includes, but is not limited to, captopril, enalapril, lisinopril, perindopril, and ramipril. The angiotensin II receptor blocker (ARB) includes, but is not limited to, losartan, candesartan, valsartan, irbesartan, telmisartan, eprosartan, olmesartan, and azilsartan. The combination may be administered separately or within the same pharmaceutical composition.

It is to be understood that the methods of the present invention may use 1-(2-ethoxyethyl)-5-(ethyl(methyl)amino)-7-((4-methylpyridin-2-yl)amino)-N-(methylsulfonyl)-1H-pyrazolo[4,3-d]pyrimidine-3-carboxamide in solution, as a suspension, as the amorphous solid or as a crystalline solid wherein the crystalline solid includes polymorphs, hydrates, solvates or combinations thereof. In particular, the present invention contemplates the use of polymorph Forms A, B and C as disclosed in US 2008/0194591. Preferred polymorphs are Forms B and C, or a combination thereof. The most preferred polymorph is Form C.

### DEFINITIONS

The term “Example 1” as used herein means 1-(2-ethoxyethyl)-5-(ethyl(methyl)amino)-7-((4-methylpyridin-2-yl)amino)-N-(methylsulfonyl)-1H-pyrazolo[4,3-d]pyrimidine-3-carboxamide or a pharmaceutically acceptable salt thereof.

The term “chronic kidney disease or CKD” includes stages 1-5 unless otherwise noted herein. It is to be understood that the present invention contemplates treating or preventing the progression of all five stages of CKD.

The term “patient” as used herein means a human.

The term “pharmaceutically acceptable salt” as used herein means those salts which are, within the scope of sound medical judgement, suitable for use in contact with the tissues of patients and lower animals without undue toxicity, irritation, allergic response and the like and are commensurate with a reasonable benefit/risk ratio. Pharmaceutically acceptable salts are well-known in the art. For example, S. M. Berge et al. describe pharmaceutically acceptable salts in detail in Berge et al., J. Pharmaceutical Sciences, 1977, 66: 1-19. The salts can be prepared in situ during the final isolation and purification of Example 1 of the present invention or separately by reacting the free base of Example 1 with a suitable organic or inorganic acid. Representative acid addition salts include, but are not limited to acetate, adipate, alginate, citrate, aspartate, benzoate, benzenesulfonate, bicarbonate, bisulfate, butyrate, camphorate, camphorsufonate, citrate, digluconate, glycerophosphate, hemisulfate, heptanoate, hexanoate, fumarate, hydrochloride, hydrobromide, hydroiodide, 2-hydroxyethansulfonate (isethionate), lactate, maleate, methanesulfonate, nicotinate, 2-naphthalenesulfonate, oxalate, pamoate, pectinate, persulfate, 3-phenylpropionate, phosphate, picrate, pivalate, propionate, succinate, sulphate, tartrate, thiocyanate, and p-toluenesulfonate.

The present invention also provides pharmaceutical compositions which comprise Example 1 formulated together with one or more non-toxic pharmaceutically acceptable carriers. The pharmaceutical compositions may be specially formulated for oral administration in solid or liquid form, for parenteral injection, or for rectal administration.

The term “pharmaceutically acceptable carrier” as used herein means a non-toxic, inert solid, semi-solid or liquid filler, diluent, encapsulating material or formulation auxiliary of any type. Some examples of materials which can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols; such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. The present invention provides pharmaceutical compositions which comprise Example 1 formulated together with one or more non-toxic pharmaceutically acceptable carriers. The pharmaceutical compositions can be formulated for oral administration in solid or liquid form, for parenteral injection or for rectal administration.

The pharmaceutical compositions of this invention can be administered to patients orally, parenterally, intraperitoneally, topically (as by powders, ointments or drops), bucally or as an oral or nasal spray. The term “parenterally,” as used herein, refers to modes of administration which include intravenous, intramuscular, intraperitoneal, intrasternal, subcutaneous, intraarticular injection and infusion.

Pharmaceutical compositions of this invention for parenteral injection comprise pharmaceutically acceptable sterile aqueous or nonaqueous solutions, dispersions, suspensions or emulsions and sterile powders for reconstitution into sterile injectable solutions or dispersions. Examples of suitable aqueous and nonaqueous carriers, diluents, solvents or vehicles include water, ethanol, polyols (propylene glycol, polyethylene glycol, glycerol, and the like), suitable mixtures thereof, vegetable oils (such as olive oil) and injectable organic esters such as ethyl oleate. Proper fluidity may be maintained, for example, by the use of a coating such as lecithin, by the maintenance of the required particle size in the case of dispersions, and by the use of surfactants.

These compositions may also contain adjuvants such as preservative agents, wetting agents, emulsifying agents, and dispersing agents. Prevention of the action of microorganisms may be ensured by various antibacterial and antifungal agents, for example, parabens, chlorobutanol, phenol, sorbic acid, and the like. It may also be desirable to include isotonic agents, for example, sugars, sodium chloride and the like. Prolonged absorption of the injectable pharmaceutical form may be brought about by the use of agents delaying absorption, for example, aluminum monostearate and gelatin.

In some cases, in order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Suspensions, in addition to Example 1, may contain suspending agents, as, for example, ethoxylated isostearyl alcohols, polyoxyethylene sorbitol and sorbitan esters, microcrystalline cellulose, aluminum metahydroxide, bentonite, agar-agar, tragacanth, and mixtures thereof.

If desired, and for more effective distribution, Example 1 can be incorporated into slow-release or targeted-delivery systems such as polymer matrices, liposomes, and microspheres. They may be sterilized, for example, by filtration through a bacteria-retaining filter or by incorporation of sterilizing agents in the form of sterile solid compositions, which may be dissolved in sterile water or some other sterile injectable medium immediately before use.

Example 1 can also be in micro-encapsulated form, if appropriate, with one or more pharmaceutically acceptable carriers as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms Example 1 can be admixed with at least one inert diluent such as sucrose, lactose, or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of such composition that they release the active ingredient(s) only, or preferentially, in a certain part of the intestinal tract in a delayed manner. Examples of embedding compositions which can be used include polymeric substances and waxes.

Injectable depot forms are made by forming microencapsulated matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides) Depot injectable formulations are also prepared by entrapping the drug in liposomes or microemulsions which are compatible with body tissues.

The injectable formulations can be sterilized, for example, by filtration through a bacterial-retaining filter or by incorporating sterilizing agents in the form of sterile solid compositions which can be dissolved or dispersed in sterile water or other sterile injectable medium just prior to use.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic, parenterally acceptable diluent or solvent such as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Solid dosage forms for oral administration include capsules, tablets, pills, powders, and granules. In such solid dosage forms Example 1 is mixed with at least one inert pharmaceutically acceptable carrier such as sodium citrate or calcium phosphate and/or a) fillers or extenders such as starches, lactose, sucrose, glucose, mannitol, and salicylic acid; b) binders such as carboxymethylcellulose, alginates, gelatin, polyvinylpyrrolidinone, sucrose, and acacia; c) humectants such as glycerol; d) disintegrating agents such as agar-agar, calcium carbonate, potato or tapioca starch, alginic acid, certain silicates, and sodium carbonate; e) solution retarding agents such as paraffin; f) absorption accelerators such as quaternary ammonium compounds; g) wetting agents such as cetyl alcohol and glycerol monostearate; h) absorbents such as kaolin and bentonite clay; and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract in a delayed manner. Examples of embedding compositions which can be used include polymeric substances and waxes.

Liquid dosage forms for oral administration include pharmaceutically acceptable emulsions, microemulsions, solutions, suspensions, syrups and elixirs. In addition to Example 1, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, and perfuming agents.

Actual dosage levels of Example 1 in the pharmaceutical compositions of this invention can be varied so as to obtain an amount of Example 1 which is effective to achieve the desired therapeutic response for a particular patient, compositions, and mode of administration. The selected dosage level will depend upon the activity of Example 1, the route of administration, the severity of the condition being treated, and the condition and prior medical history of the patient being treated.

The total daily dose of Example 1 administered to a patient is 0.3 to 400 mgs. If desired, the effective daily dose can be divided into multiple doses for purposes of administration, e.g. two to four separate doses per day.

### Synthetic Preparation

### Example 1

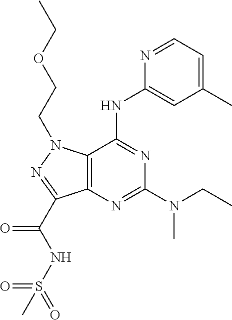 [SMILES: CCOCCn1nc(C(=O)NS(C)(=O)=O)c2nc(N(C)CC)nc(Nc3cc(C)ccn3)c21]

### 1-(2-ethoxyethyl)-5-(ethyl(methyl)amino)-7-((4-methylpyridin-2-yl)amino)-N-(methylsulfonyl)-1 H-pyrazolo[4,3-d]pyrimidine-3-carboxamide

The title compound was prepared as described in U.S. Pat. No. 7,572,799 (see Example 115). Polymorph Forms A, B, and C of the title compound were prepared as described in U.S. Published Patent Application No. 2008/0194591 (U.S. patent application Ser. No. 11/913,091). U.S. Pat. No. 7,572,799 and US 2008/0194591 are hereby incorporated by reference.

### Biology/Pharmacology

In single dose toxicity studies in mice and rats, no deaths were observed; the maximum non-lethal dose was 2000 mg/kg. In dogs, doses up to 1000 mg/kg were given and no adverse effects were noted.

In humans, Example 1 was evaluated in single dose and multiple dose clinical studies in 46 healthy male volunteers aged 21 to 49 years. Both single and multiple dose clinical studies were conducted with an oral solution or suspension ranging from single doses of 0.3 to 400 mg and multiple doses of 30 to 200 mg. Example 1 was rapidly absorbed in humans following single doses of solution with time of occurrence of Cₘₐₓ (Tₘₐₓ) of 1.1 to 1.5 hours. The terminal half-life did not alter significantly with dose and ranged from 11.9 to 15.7 hours. Following multiple doses, Example 1 was rapidly absorbed (Tₘₐₓ of 1.1 to 3.5 hours) and t½ on days 1 and 13 ranged from 11.6 to 14.5 hours. Single doses of Example 1 were well tolerated, no serious adverse events occurred. In the multiple dose study, Example 1 was well tolerated over the dose range of 30 to 200 mg. No significant changes in vital signs, ECGs, laboratory safety test results, or physical examination results were reported at any dose.

TABLE 1

| Assay/Model | Example 1 Potency/Efficacy |
|---|---|
| PDE5 enzyme (human platelet) | IC₅₀ = 0.71 nM |
| PDE5 enzyme (rat platelet) | IC₅₀ = 0.93 nM |
| PDE5 enzyme (dog platelet) | IC₅₀ = 0.65 nM |
| Aortic ring relaxation (rat) | EC₅₀ = 3.1 nM |
| In vivo SHR (oral dosing) | ECₘₐₓ = 6.5 nM |
| | (unbound plasma concentration) |
| PDE6 enzyme (human retinal cone) | IC₅₀ = 28.9 nM |
| PDE6 enzyme (human retinal rod) | IC₅₀ = 63.6 nM |
| PDE11 enzyme (human) | IC₅₀ = 26.3 nM |

IC50 = 50% inhibitory concentration;
EC50 = efficacious concentration;
ECmax = maximally efficacious concentration;
SHR = spontaneously hypertensive rat.

Example 1 is a competitive inhibitor of human PDE5, with a mean IC₅₀ value of 0.71 nM (0.34 ng/mL) using platelet-derived enzyme. Potencies against rat and dog platelet-derived PDE5 are similar at 0.93 and 0.65 nM, respectively. Example 1 has PDE6 IC₅₀ potencies against human retinal cone and rod of 28.9 nM (41-fold selectivity) and 63.6 nM (90-fold selectivity), respectively. The IC₅₀ of Example 1 against human PDE11 is 26.3 nM (37-fold selectivity). Greater than 1000-fold selectivity exists relative to PDE enzymes 1, 2, 3, 4A, 4B, 4D, 7B, 8A, 9, and 10.

The direct functional effects of Example 1 were demonstrated in isolated rat aortic rings. Example 1 induced aortic ring vasorelaxation with a mean EC₅₀ value of 3.1 nM.

The antihypertensive efficacy of Example 1 was assessed following daily oral dosing in conscious, spontaneously hypertensive rats (SHR) monitored continuously by radiotelemetry. A significant reduction in mean arterial pressure (MAP) was achieved at free plasma concentrations corresponding to approximately 7-fold the rat PDE5 IC₅₀. Treatment of SHR with Example 1 induced a significant, sustained reduction in MAP over a 14-day dosing period. Similarly, treatment with the ACE inhibitor enalapril induced a significant, sustained reduction in MAP over a 14-day dosing period. When combined with an ACE inhibitor, Example 1, afforded greater MAP lowering effects than the ACE inhibitor alone.

Example 1 was assessed in a series of safety pharmacology studies outlined in Table 2. For in vivo studies, the oral route of exposure was used. Rats given diazepam or frusemide served as positive controls in the locomotor activity and fluid/electrolyte excretion studies, respectively. Dofetilide was used for assay validity or a positive control in the dofetilide and hERG assays, respectively. Positive controls or animals given positive controls were not assessed concurrently in the remaining studies as these studies utilized well-characterized models to evaluate safety parameters.

TABLE 2

| Study | Concentration or Dose | |
|---|---|---|
| [³H]dofetilide binding assay | 0.003-100 | μM |
| hERG assay | 3 and 10 | μM |
| Purkinje fiber assay | 0.01, 0.1, 1, 10 | μM |
| In vivo toleration - rat | 100, 300, 1000 | mg/kg |
| Appearance/behavior - single dose | | |
| Central nervous system - rat | 3, 30, 300 | mg/kg |
| Locomotor activity | | |
| Renal - rat | 3, 30, 300 | mg/kg |
| Fluid and electrolyte excretion | | |
| Pulmonary function - rat | 3, 30, 300 | mg/kg |
| Respiratory parameters | | |
| Cardiovascular - anesthetized dog | 1.47-387 | μg/kg/min |
| PK/PD evaluation with monitoring | | |
| Cardiovascular - conscious dogs | 0.5, 1.5, 5 | mg/kg |
| Hemodynamic and ECG parameters | | |

No relevant effects were noted in the hERG patch clamp assay, the dog Purkinje fiber assay, or the [³H]dofetilide-binding assay up to 10 μM (4.77 μg/mL). Example 1 competitively displaced the [³H]dofetilide by 5.5% at 30 μM (14.3 μg/mL and 32.5% at 100 μM (47.7 μg/mL). The no-effect concentration of 4.77 μg/mL is approximately 426 times the Cₘₐₓ (˜0.011 μg/mL as free fraction) based on human pharmacokinetics (PK) obtained after a 30 mg dose, which was well tolerated, suggesting a low potential for QT prolongation.

Results in rats given up to 300 mg/kg of Example 1 demonstrated minor, nondose-related effects consisting of decreased rearing and center rearing, but no adverse effects on pulmonary function. Example 1 demonstrated a dose-related decrease in urine volume and electrolyte excretion consistent with results from other PDE5 inhibitors.

In dogs, Example 1 at 0.5, 1.5, and 5 mg/kg induced small, but significant reductions in blood pressure and left ventricular end-diastolic pressure. These cardiovascular effects are consistent with cGMP elevation in vascular smooth muscle, resulting from inhibition of cGMP-specific PDE5. Example 1 was not associated with any relevant effects on ECG parameters in dogs. No clinically-relevant effects on ECG parameters were noted in humans given single and multiple doses of Example 1. Cₘₐₓ in dogs at 5 mg/kg was 12.8 mg/mL or ˜8 times higher than the Cₘₐₓ (1.57-1.63 μg/mL) determined in humans after a 30 mg dose, which was well tolerated.

Liguid chromatography/mass spectrometry was used to determine Example 1 concentrations in plasma samples from PK and toxicokinetic studies in rats, dogs, and humans. For toxicokinetic studies, this method was validated over a concentration range of 10 to 1000 ng/mL for a 50 μL plasma sample. Radiometric methods were used to measure [¹⁴C] Example 1 derived radioactivity in biological samples from the in vitro and in vivo metabolism studies.

Absolute oral bioavailability of Example 1 following a single dose was 82% in rats and 77% in dogs. Following intravenous (IV) administration of Example 1, plasma clearance in rats and dogs was lower that the liver-blood flow in the corresponding species, indicating that Example 1 is a low-clearance compound. The steady-state distribution volume was lower than total body water in the rat and dog.

In a tissue distribution study in rats given radiolabelled Example 1, as substantial portion of the dose remained within the gastrointestinal tract and systemic distribution was generally in proportion to tissue-blood flow. Plasma protein binding values for Example 1 were determined to be 99.3% in rat, dog, and human and 98.8% in rabbit plasma.

In rats and dogs, greater than 80% of the administered doses (total radioactivity) were eliminated in feces. The majority of the radioactivity was recovered within 48 hours. Total radioactivity recovered in rats and dogs was greater than 92%.

The potential for Example 1 to inhibit CYP1 A2, CYP2C9, CYP2C19, CYP2D6 and CYP3A4 (midazolam, testosterone and felodipine) was determined in human liver microsomes. No inhibition was observed with IC₅₀ values >30 μM.

There were no deaths, clinical signs, or effects on body weight, and no pathologic findings in mice or rats given single oral doses of Example 1 at 20, 200, or 2000 mg/kg and observed for 14 days. No toxicity was observed in rats given single oral doses of 100, 300, or 1000 mg/kg, whereas mild gastrointestinal effects occurred in dogs given single-oral doses of 100, 500, o 1000 mg/kg.

Micronized Example 1 (polymorph form C) was assessed in single-dose toxicity/toxicokinetic studies in rats and dogs to compare exposure with polymorph B. The micronized Example 1 polymorph form C enabled the administration of higher doses in rats and dogs than was achievable with polymorph form B. At equivalent doses of polymorph forms B and C (100 mg/kg in dogs and 100 and 300 mg/kg in rats), there were no differences in systemic exposure between the polymorph forms.

Example 1 was assessed in a series of genetic toxicology assays consisting of the microbial reverse mutation, in vitro cytogenetic (human lymphocyte), and in vivo rat micronucleus assays. Study designs and dose selection were consistent with International Conference on Harmonization and Organization for Economic Cooperation and Development guidelines for mutagenicity and clastogenicity assays. All in vitro tests were conducted with and without exogenous metabolic activation using concentrations up to those limited by cytotoxicity or insolubility. Example 1 was not genotoxic in either in vitro or in vivo assays (Table 3).

TABLE 3

| Test System | Dose | Result |
|---|---|---|
| Mutagenicity: | 50-5000 mg/plate (±S9) | Negative |
| Salmonella typhimurium | | |
| (Strains TA-1535, TA-1537) and | | |
| Escherichia coli | | |
| (Strain WP2uvrA) | | |
| Clastogenicity In Vitro: | 100, 350, 500, μg/mL (3 h- | Negative |
| Structural Chromosome | S9) | |
| Aberration in Human | 100, 200, 350, μg/mL | |
| Peripheral Lymphocytes | (3 h + S9) | |
| | 12.5, 50, 100, μg/mL (24 h- | |
| | S9) | |
| Clastogenicity In Vitro | VC, 100, 300, 600 mg/kg/ | Negative |
| Micronucleus Assay in Rat Bone | day | |
| Marrow | | |

S9 = Postmitochondrial supernatant from livers of rats treated with Aroclor 1254;
VC = Vehicle control.

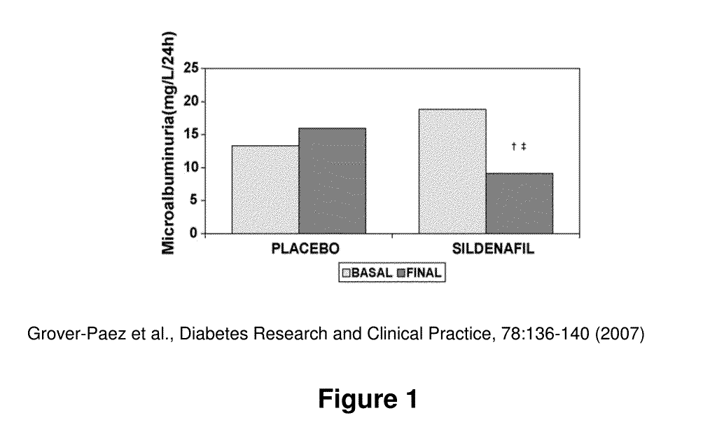

The PDE5 inhibitor sildenafil reduces albuminuria in Type II diabetic patients with early-stage diabetic nephropathy (FIG. 1). This data supports the use of PDE5 inhibitors for treating DN and/or CKD that have a suitable pharmacokinetic (PK) and safety profile in humans. Example 1 is a potent and selective PDE5 inhibitor that was well tolerated in humans following oral administration. When compared to sildenafil, Example 1 appears to be a more potent PDE5 inhibitor with greater selectivity for PDE5 over PDE6. Further, Example 1 has a 3-4 fold longer half-life in humans (Table 4). These advantages make Example 1 superior to sildenafil for treating or delaying the progression of albuminuria, DN and/or CKD in humans, particularly diabetic patients.

TABLE 4

| Criteria | Example 1 | Sildenafil |
|---|---|---|
| PDE5 IC₅₀ (nM) | 0.71 | 4* |
| PDE5/PDE6 selectivity | 41 fold | 9 fold* |
| Human Half-life (hr) | 11.9 to 15.7 | 3.7 |

*As reported in Ballard et al., The Journal of Urology, 159: 2164-2171, June 1998

We claim:

1. A method of treating macroalbuminuria in a patient comprising the step of administering to the patient, in need of such treatment, a therapeutically effective amount of a combination of (i) 1-(2-Ethoxyethyl)-5-(ethyl(methyl)amino)-7-((4-methylpyridin-2-yl)amino)-N-(methylsulfonyl)-1H-pyrazolo[4,3-d]pyrimidine-3-carboxamide, or a pharmaceutically acceptable salt thereof, and (ii) an angiotensin converting enzyme inhibitor.

2. The method of claim 1 wherein the combination is administered together.

3. The method of claim 1 wherein the combination is administered sequentially.

4. A pharmaceutical composition comprising (i) 1-(2-Ethoxyethyl)-5-(ethyl(methyl)amino)-7-((4-methylpyridin-2-yl)amino)-N-(methylsulfonyl)-1H-pyrazolo[4,3-d]pyrimidine-3-carboxamide, or a pharmaceutically acceptable salt thereof, (ii) an angiotensin converting enzyme inhibitor, and (iii) at least one pharmaceutically acceptable carrier, diluent, or excipient.

5. A method of treating macroalbuminuria in a patient comprising the step of administering to the patient, in need of such treatment, a therapeutically effective amount of the pharmaceutical composition of claim 4.

6. A method of treating macroalbuminuria in a patient comprising the step of administering to the patient, in need of such treatment, a therapeutically effective amount of a combination of (i) 1-(2-Ethoxyethyl)-5-(ethyl(methyl)amino)-7-((4-methylpyridin-2-yl)amino)-N-(methylsulfonyl)-1H-pyrazolo[4,3-d]pyrimidine-3-carboxamide, or a pharmaceutically acceptable salt thereof, and (ii) an angiotensin receptor blocker.

7. The method of claim 6 wherein the combination is administered together.

8. The method of claim 6 wherein the combination is administered sequentially.

9. A pharmaceutical composition comprising (i) 1-(2-Ethoxyethyl)-5-(ethyl(methyl)amino)-7-((4-methylpyridin-2-yl)amino)-N-(methylsulfonyl)-1H-pyrazolo[4,3-d]pyrimidine-3-carboxamide, or a pharmaceutically acceptable salt thereof, (ii) an angiotensin receptor blocker, and (iii) at least one pharmaceutically acceptable carrier, diluent, or excipient.

10. A method of treating macroalbuminuria in a patient comprising the step of administering to the patient, in need of such treatment, a therapeutically effective amount of the pharmaceutical composition of claim 9.
